(19) United States
(12) Patent Application Publication
(10) Pub. No.: US 2005/0220835 A1
(43) Pub. Date: Oct. 6, 2005

(54) Agent eluting bioimplantable devices and polymer systems for their preparation

(76) Inventors: Ramesh Babu Jayaraman, Fremont, CA (US); Russell Gill, Emeryville, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/813,315

(22) Filed: Mar. 30, 2004

(51) Int. Cl. A61F 2/00

(52) U.S. Cl. 424/423

(57) ABSTRACT

Bioimplantable devices comprising a polyetherurethane modified by admixture with siloxane surface modifying additive that may be loaded with a therapeutic agent are provided.

### FIELD OF THE INVENTION

The invention is concerned with bioimplantable devices which are adapted for the site specific elution of biologically active materials, such as pharmaceutical compositions. The invention is also directed to the novel bioactive agent loading of polymers, particularly certain polyurethane polymers and to the fabrication of bioimplantable devices including such loaded polymer systems.

### BACKGROUND OF THE INVENTION

The loading of polymers with certain biologically active agents has been studied somewhat. Use of implantable medical devices containing polymer loaded with therapeutic agents can provide a local alternative to systemic administration of agents. Among the benefits of such local treatment are that it enables disease to be treated by agents and in dosages of such agents that are not suitable for systemic therapy. Such a benefit is often, but not necessarily, in addition to the basic intervention that the medical device is designed to achieve.

A common site of medical intervention with agent loaded polymer medical devices is the vascular system. Placement of central venous catheters, arterial and intravenous catheters, and so forth may be performed to obtain medical data such as blood pressure or to provide local or systemic delivery of therapeutic agents. Placement of vascular patches, arterial and venous stents and stent-grafts, grafts, and so forth may be performed to correct an underlying anatomic abnormality and/or to deliver therapeutic agents.

Researchers have studied the delivery of therapeutic agents via methods including infusion, coatings, and structural modifications such as reservoirs. Therapeutic agents may be targeted at conditions such as infection, vascular hyperplasia, restenosis, and neoplasia.

U.S. Pat. No. 6,585,995 teaches treatment and inhibition of vaso-occlusive events through the use of an anti-platelet agent administered parenterally and by a sustained release device that may be used during a surgical procedure. Chen et al., Recombinant Mitotoxin Basic Fibroblast Growth Factor-Saporin Reduces Venous Anastomotic Intimal Hyperplasia in the Arteriovenous Graft, Circulation. 1996;94:1989-1995, describes femoral arteriovenous grafts with local infusion devices attached to an osmotic pump that can deliver therapeutic agents directly through the wall of the graft.

U.S. Pat. No. 6,273,913 describes a stent design that includes channels that may contain therapeutic agents (i.e. rapamycin). Such channels allow targeted delivery of agents that inhibit neointimal proliferation and restenosis. Cordis also discloses local delivery of therapeutic agents from the struts of a stent and the mixture of agent and polymer to hold the agent to the stent.

U.S. Pat. No. 6,599,928 discloses intravascular stents—biodegradable, plastic and metal stents—and a coating allowing sustained release of cytostatic agent. U.S. Pat. No. 4,459,252 discloses a polymeric vascular graft with porous surfaces in communication with a hollow interior through which substances may be released by slow, sustained release. U.S. Pat. No. 6,440,166 teaches a multi-layered vascular graft with a non-thrombogenic layer formed by chemically binding a non-thrombogenic agent to PTFE or a polyurethane polymer.

U.S. Pat. No. 6,589,546 teaches multi-layered implantable medical devices containing a barrier layer that enables controlled release of a bioactive agent. This patent also teaches coating of the medical device with a bioactive agent. U.S. Patent Application 2002/0107330 teaches delivery of a therapeutic agent from a medical device composed of block copolymer that is loaded with a therapeutic agent.

These devices and techniques have had limited success. Significant limitations of the above delivery systems include, inter alia, the need for additional barrier layers to control agent release, the lack of porosity in certain polymers, and the inability to deliver multiple agents separately. The present invention provides improvements in these areas. In accordance with one aspect of the invention, biologically active agents can be delivered in a highly site specific fashion through implantable devices hereof such that undesired, systemic exposure to the active agents is minimized while local, desired concentrations of the active agent are maintained. Improved therapeutic efficacy is achieved as is improved convenience and treatment flexibility.

### SUMMARY OF THE INVENTION

The invention concerns implantable devices, such as synthetic implants for anatomic support, tissue replacement or functional facilitation i.e. stents, vascular grafts, ventricular assist devices, and so forth. Such a device may be multi-layered. Such a device contains at least one region or layer for intimate tissue contact with this intimal layer or region either comprising or being in fluid communication with a portion of the device comprising a polyetherurethane. The polyetherurethane section(s) may comprise part of a layer, parts of multiple layers, or all of a layer or layers. The polyetherurethane of said layers may be the same or different. In some preferred embodiments, the devices of the invention further comprise at least one polyetherurethane portion that is modified by admixture with a siloxane surface modifying additive. At least a portion of a siloxane modified polyetherurethane section of the device contains at least one therapeutic agent.

In the case of vascular grafts, the devices of the invention may comprise a generally tubular polyetherurethane having a lumen and having two ends. The graft may further comprise an intimal layer comprising a substantially microporous polyetherurethane. In certain embodiments, the graft devices further comprise at least one intermediate layer comprising a substantially nonporous polyetherurethane and an adventitial layer comprising a substantially microporous polyetherurethane. A polyetherurethane portion of at least one layer is preferably modified by admixture with a siloxane surface modifying additive. At least a portion of at least one layer contains at least one therapeutic agent. In certain preferred embodiments, at least a part of the siloxane modified polyetherurethane portion of at least one layer contains the agent.

The invention also concerns methods of forming prosthetic grafts containing polyetherurethane and a therapeutic agent comprising contacting a prosthetic graft containing a polyetherurethane with a solution comprising a solvent and said therapeutic agent for a period of time sufficient to load said graft with a desired amount of therapeutic agent. Preferably, the solvent substantially swells the polymer allowing the agent to diffuse into the polymer structure or matrix while said polyetherurethane is substantially insoluble in said solvent.

Another aspect of the invention concerns methods for forming prosthetic grafts which include one or more bioactive, preferably therapeutic, agents. Some preferred embodiments comprise mixing said agent with a polyetherurethane polymer, manufacturing the device; applying the polymer to a surface of the device or causing the layer or layers to be formed from such polymer. Another aspect of the invention provides methods for forming a coating containing polyetherurethane polymer with siloxane based surface additives, said polymer loaded with a therapeutic agent. The invention also concerns biocompatible devices comprising a blend of polyetherurethane polymer with siloxane based surface modifying additive, said blend being loaded with at least one therapeutic agent.

Another aspect of the invention is the provision of devices comprising a polyetherurethane having one or more layers, at least part of one layer comprising an admixture of siloxane surface modifying additive, and at least part of a layer comprising one or more therapeutic agents.

### DESCRIPTION OF THE DRAWINGS

FIG. 1 graphically depicts experimental data demonstrating the release profile of Rapamycin from a vascular access graft (in saline).

FIG. 2 graphically depicts experimental data demonstrating the release profile of Paclitaxel from a vascular access graft (in saline).

FIG. 3. graphically depicts experimental data demonstrating the distribution of rapamycin at the rings of a stent-graft.

FIG. 4: graphically depicts experimental data demonstrating the release profile for rapamycin from a stent-graft (in bovine serum albumin).

FIG. 5 graphically depicts experimental data demonstrating the release profile of Paclitaxel from film (in bovine serum albumin).

### DETAILED DESCRIPTION OF PREFERRED EMBODIMENTS

This invention relates to the loading of a polymer bioimplantable device with one or more agents, whereby the agent may be delivered either locally or systemically and multiple agents may be delivered either in combination or separately.

Loading the device of the invention with therapeutic agent(s) provides an important additional mechanism for therapy and treatment. Devices of the invention may improve the bioavailability of an agent. Devices of the invention may be loaded with agents that are toxic, ineffective, poorly tolerated, poorly absorbed, or contraindicated when administered through other means, such as by oral administration. Devices of the invention may also be used to administer dosage amounts that would be unsuitable for systemic therapy. For example, many agents administered systemically to treat one body or organ system, cause adverse effects in other body or organ systems. Such adverse effects may limit the dosage amount, length of time, effectivity, and so forth. The bioimplantable device of the invention may be used to target the particular system, organ, disease, and so forth for delivery of agent(s).

Additionally, loading of such devices may provide more rapid treatment and greater predictability of availability. Besides improving treatment, such mechanisms may save health care costs. For example, loading of a vascular graft with rapamycin for treatment of vascular hyperplasia at the anastomosis site enables the rapamycin to be released in close proximity to anastomotic sites. Such local delivery may serve as the sole treatment or as an adjunct to other treatments. An additional feature of the invention is that such bioimplantable devices may be designed for systemic therapy or non-local delivery as well.

The devices of the invention contain at least one polyetherurethane polymer that is modified by admixture with a siloxane surface modifying additive. Certain suitable polymers are found in U.S. Pat. Nos. 4,861,830 and 4,675,361, the disclosures of which are incorporated herein in their entirety. One example is the commercially available polymer Thoralon® which is marketed by Thoratec Corporation. In some preferred embodiments the polyetherurethane polymer of at least one layer or region comprises at least about 1 percent by weight of a polysiloxane polyurethane copolymer surface modifying agent; more preferably 1 to about 40 percent by weight; and most preferably 1 to about 5 percent by weight.

The polymer may be loaded in whole, in part, or in select segments with a therapeutic agent by dissolving the agent in a common solvent for the polymer as well as the therapeutic agent. The polymer may be loaded before or after fabrication into a device. In certain preferred embodiments it is preferable to load the polymer after fabrication of the device to avoid loss of agent during the fabrication process.

Suitable solvents for the polymer include highly polar solvents like dimethyl acetamide, dimethyl formamide and N-methyl pyrrolidone. Suitable solvents also include tetrahydrofuran. Methods known to those of ordinary skill may be used to load the polymer with the agent. One such method is the swelling technique described in U.S. Patent Application 20020107330, the disclosure of which is incorporated herein in its entirety. In this technique, an agent or combination of agents is dissolved in a solvent that is non-solvent for the polymer. The polymer is soaked in the solvent containing agent(s) for an appropriate period of time. In some embodiments, the polymer is soaked until equilibrium is established.

In some embodiments, the solvent swells the polymer allowing agent(s) to infuse into the polymer. After equilibrium is established, the polymer is removed from the solvent and residual solvent may be removed by heating or under vacuum, conditions which allow agent(s) to remain incorporated on the polymer matrix.

Such loading techniques may be repeated as necessary to load additional agents. These techniques may also be repeated with additional (either the same or a different) polymer to allow agents to be loaded in combination or loaded on the polymer without contacting one another and maintained separately. Agent(s) may be loaded together or loaded separately. Agents may also be loaded separately but allowed to contact one another once loaded. The agents loaded in each instance may have the same or different therapeutic uses. The agents may also be mixed together and then loaded.

A particular section of a device may be loaded by selectively sealing off the section appropriately and then contacting the agent containing solution with the section to be loaded. The solvent swells only the isolated section in which an agent is to be loaded. As the solvent evaporates and the polymer returns to its original shape the dissolved agent is left behind. The agent is physically trapped into the matrix of the polymer section and/or physically adsorbed on the surface. This distribution will depend on the agent-polymer interaction and the solvent used to swell the polymer. In other embodiments a particular section of the device may be loaded by fluid communication with another section of the device.

The polymer structure may be cast or molded according to methods known to those of ordinary skill into a variety of shapes, layers, segments, divisions and so forth suitable to match the physical property needs of the device, the release profile desired for the agents, the target site, and so forth. Devices that may be crafted include but are not limited to the following: tissues, anatomical supports, arterio-venous shunts, stents, stent-grafts, grafts, balloons, sheaths, catheters, percutaneous leads, cannulae, vascular and cardiac patches, wound healing patches, prosthetic ligaments, prosthetic tendons, prosthetic vertebral discs, coatings and so forth.

Such devices may be composed of single or multiple polymer-agent complexes that may be either the same or different. When a plurality of agents is loaded on a device such plurality may include different therapeutic agents or separate agent-polymer complexes of the same agent or a combination of both. The devices may also be structured into layers or segments with varying properties such as porosity; pore size; siloxane content; agent related factors such as concentration, total load, chemical structure, polarity, molecular weight and so forth. Varying these factors varies the chemical and/or physical properties of the device. For example, using polymers with varying porosity or pore size alters the permeability characteristics of the device. If multiple agents are used the agents may be maintained separately by polymers with low porosities or polymers loaded with a different agent. In other preferred embodiments multiple complexes of the same agent may be maintained separately by polymer with low porosities or polymer loaded with a different agent. Porosity would also affect both agent loading and release.

The devices may also be combined with other polymeric devices. Polymer devices available commercially include the multilayer Vectra® vascular dialysis graft described in U.S. Pat. No. 4,604,762, No. 4,731,073, No. 4,675,361, No. 4,861,830. The fields of intervention for such devices include but are not limited to vascular, genitourinary, nephrologic, pulmonary, cardiovascular, dermatologic, orthopedic, and so forth.

The therapeutic agents include any agent that may be administered to the organism. Such agent(s) will usually be designed for local delivery, but may also be provided for systemic and non-local delivery. Such agent(s) may be of any release type including immediate release, sustained release, or controlled release as the material porosity or loading technique may be altered by methods known to those of ordinary skill.

The therapeutic agent(s) may be any pharmaceutical, chemical, or biological agent that is soluble and stable in the polymer solvent. Suitable solvents would be known to a person of skill in the art, for example, tetrahydrofuran. Suitable polymer solvents for Thoralon® include dimethyl acetamide, dimethylformamide and N-methylpyrrolidone. Agent(s) may be determined by methods known to those of ordinary skill and include anti-platelet; anti-stenotic; anti-hyperplasia; anti-thrombotic, anti-proliferative; anti-migratory; anti-fibrotic; angiogenic; agents affecting extracellular matrix production and organization; anti-neoplastic; anti-mitotic agent; anti-coagulant; vascular cell growth promoter; vascular cell growth inhibitor; vasodilating agent; an agent that interferes with endogenous vasoactive mechanism; antibiotic; anti-fungal; anti-bacterial; anti-septic; anesthetic; anti-inflammatory; wound healing; fibroplastic; pro-inflammatory; chemotactic; steroid; neurologic; psychiatric; chemotherapeutic; steroidal; palliative; radiologic agent; contrast agent, as well as any agent or combination of agents that may be administered to the organism.

The amount of an agent loaded would depend on multiple factors including the agent mechanism of action, solubility, release rate, target site, effective concentration, and so forth. Loading may also be effected by varying devices; device portions or layers; agents; or therapies. The loading may be measured in a portion of a layer, a layer, combination of portions and/or layers, or the device as a whole. The loading capacity for an agent soluble in the polymer solution ranges from about 0.001 to 40 weight percent of the siloxane modified polyetherurethane; preferably about 0.001 to 30 weight percent of the siloxane modified polyetherurethane; more preferably about 0.001 to 20 weight percent of siloxane modified polyetherurethane; still more preferably about 0.001 to 10 weight percent of siloxane modified polyetherurethane; and still more preferably about 0.001 to 5 weight percent of siloxane modified polyetherurethane.

The loading capacity may also be of an amount less than a systemically effective amount. Once again loading may be effected as detailed above. The device may be loaded preferably in an amount less than a systemically effective amount; preferably an amount less than about 50% of a systemically effective amount by weight of the composition; more preferably an amount less than about 40% of a systemically effective amount by weight of the composition; more preferably an amount less than about 30% of a systemically effective amount by weight of the composition; more preferably an amount less than about 20% of a systemically effective amount by weight of the composition; more preferably an amount less than about 10% of a systemically effective amount by weight of the composition; more preferably an amount less than about 5% of a systemically effective amount by weight of the composition; still more preferably an amount less than about 1% of a systemically effective amount by weight of the composition.

The loading capacity may also be of an amount greater than a systemically effective amount. Once again loading may be effected as detailed above. In addition to the factors discussed above such loading would be dependent on target site, release rate, toxicities, and so forth. In some embodiments the agent may be loaded in an amount 10% greater than a systemically effective amount by weight of the composition. Such loading of greater than systemically effective amounts may be valuable in multiple areas such as the delivering of toxic agents to treat cancer or treatment of obstructive diseases like tracheo-bronchial obstruction.

The release profile of an agent-polymer complex may be determined following loading. One method is using high performance liquid chromatography with comparison to control to determine the release of agent from polymer over time. Other methods known in the art may be used as well. Adjustment of multiple factors including polymer porosity, agent concentration within polymer, and so forth may be used to alter the release profile for a particular agent.

The following are provided by way of example and not as limitations.

One preferred embodiment that would illustrate the versatility of the multi-agent polymer structure would be a polymer vascular dialysis graft. The polymer may be configured into a vascular dialysis graft containing three layers. These layers are made of polyurethane with at least a portion of at least one layer containing a polyetherurethane modified by admixture with a siloxane surface modifying additive. In another preferred embodiment each of the layers is a polyetherurethane with at least a portion of at least one layer modified by admixture with a siloxane surface modifying additive.

The layers of a preferred embodiment are an intimal layer forming the lumen; an intermediate layer approximating the media; and attached to the intermediate layer is an adventitial layer that contacts tissue. With this structure, there exist numerous possibilities in agent loading. In some embodiments a layer may be substantially nonporous. In other embodiments a layer may be porous. Porosity may be varied so that a layer is permeable to different compounds. For example, a layer may be impermeable to blood. Another example would be a layer that is porous to low molecular weight compounds.

One or more therapeutic agents may be loaded on only the intimal layer of a graft; or on each layer of a graft; or on a combination of layers. A therapeutic agent may also be loaded onto selected sections of the graft. For example, agent may be isolated on the venous end of a dialysis access graft to impact venous stenosis of an access graft anastomosis or an agent may be loaded on the arterial end of a coronary artery bypass graft to minimize proximal ostial hyperplasia. In yet another example an agent may be incorporated in discrete bands along the length of a device to provide diffusion along the whole device without increasing the systemic agent load to toxic levels. Also multiple agents may be incorporated in different segments axially or circum-ferentially throughout the device. The end of a graft may have an anti-proliferative agent for reduction of stenosis with an anti-thrombotic agent in the center section of the inner blood contacting layer and an antibacterial agent on the outer polymer layer for infection resistance.

Many agent possibilities exist as well. For example, a porous intimal layer may be loaded with an anti-thrombotic agent and an outer porous layer could be loaded with an anti-restenotic or anti-inflammatory agent. Some preferred embodiments may contain a substantially nonporous intermediate layer, and the agents may remain separated. An alternative embodiment would be an intermediate layer that it is impermeable to blood, but may, depending on multiple factors such as porosity, still be permeable to low molecular weight compounds. In other embodiments, a porous outer adventitial layer may contain an agent for immediate release and an intermediate layer may contain an agent for sustained or controlled release.

In yet another aspect, only part of the graft, or selected segments may be loaded with agent. Such determinations might be influenced by the release profile of the agent used or the disease or target to be treated. Since restenosis at the venous anastomosis is a common problem following graft implantation, an agent or combination of agents may be loaded at the venous end of the graft. Thus, the release of the agent would occur near the venous anastomosis. If a problem at the arterial anastomosis needed to be addressed, an agent or combination of agents could be loaded at the arterial end of the graft.

Another embodiment is that an agent is loaded onto a graft starting from the venous anastomosis to a distance of about 1-10 cm in length, and in certain embodiments, about 5 cm in length. Agent may be preferentially loaded onto selected layers. In some preferred embodiments agent may be preferentially loaded onto an intimal layer and an intermediate layer.

Target sites at both ends of the graft could be treated by loading agents onto different ends of the same or different layer. The agents targeting different problems could be separated from each other by an intervening polymer segment of low porosity to the respective agents or by determining the likelihood of mixing based on polymer porosity and agent release rate.

To load on the inner layer or intimal layer of the graft, one end of a graft would be sealed and a solution of agent in a solvent would be placed inside the graft. An outer or intermediate layer bordering the inner layer of the graft may be selected so it is substantially nonporous or impermeable to the agent, solvent, or solution. The bordering layer may also be selected so it is porous. An agent may incorporate into a layer depending on factors such as the process of loading; agent used; solvent used; agent-solvent interaction and so forth. During the contact of the solution with the graft, the agent and the solvent may diffuse into the inner layer only, or the inner layer and some or all bordering layer(s). Incorporation of agent into a layer depends on factors such as the process of loading; agent used; solvent used; agent-solvent interaction and so forth. Excess solution, if present, may be drained after contacting for desired period of time and the graft may be dried to remove excess solvent. In some embodiments about all the solvent is allowed to evaporate through the solid middle layer. This method may allow one to impregnate a known quantity of the agent in the graft section.

To load agent onto the outermost or adventitial layer of a graft, a graft would again be sealed, and then immersed in a solution of an agent so that only the adventitial layer is in contact with the solution. The agent in the solvent may also be added drop wise over the adventitial layer or sprayed and the solvent allowed to evaporate. This process may be repeated several times until required amount of agent is added to the adventitial layer. It is also possible that two or more different agents may be loaded (e.g., the inner layer may contain an anti-platelet agent and the adventitial layer may contain an anti-restenosis agent or the inner layer may contain an anti-restenosis agent and the adventitial layer may contain an anti-inflammatory agent). (Such agents may have the same or different therapeutic uses.). Agents may also be mixed together and loaded into the desired layers of a graft.

After implantation, agent elutes from the graft, and depending on location may enter an adjacent artery, vein, tissue, and so forth. Such elution is preferred at therapeutic concentrations, and may be in immediate release, controlled release or sustained release forms. The agent, depending on its target site, may then act either locally, systemically, or at another desired target site.

The agent may also be dissolved in the polymer and the device may be fabricated. In some preferred embodiments, agent may be dissolved in the raw material Thoralon® and the vascular access graft fabricated. Persons of ordinary skill would consider pre- or post-fabrication loading to have advantages and disadvantages based on their preferred results. For example, pre-fabrication loading may be less desirable because of agent losses but more desirable for ease of production because the fabricated graft may undergo several processing steps to get to the finished product. Processing steps may decrease agent availability.

Another embodiment consists of a polymer-agent coating. Such a coating may be applied to devices by processes known in the art including a spray process or a dip process. After applying the coating, solvent in the polymer solution may be evaporated under suitable conditions leaving behind a film of polymer-agent. Coating may be applied to all or part of a device, and may be porous or a thin solid substantially nonporous film. Additionally, multiple coatings containing the same or different polymer-agent combinations may be applied to a device.

### EXAMPLE 1

### Vascular Access Graft Loaded with Rapamycin

A 100 ppm solution of Rapamycin (˜0.63 ml; ˜63 μg) in isopropanol was poured into an aluminum pan. Four vascular access graft sections (˜3×6 mm each; ˜30 mg) were deaired in the solution. All of the solution was absorbed. The vascular access graft pieces (˜0.05% loading w/w vascular access graft) were transferred to a new pan and air dried for 60 minutes at 80° C.

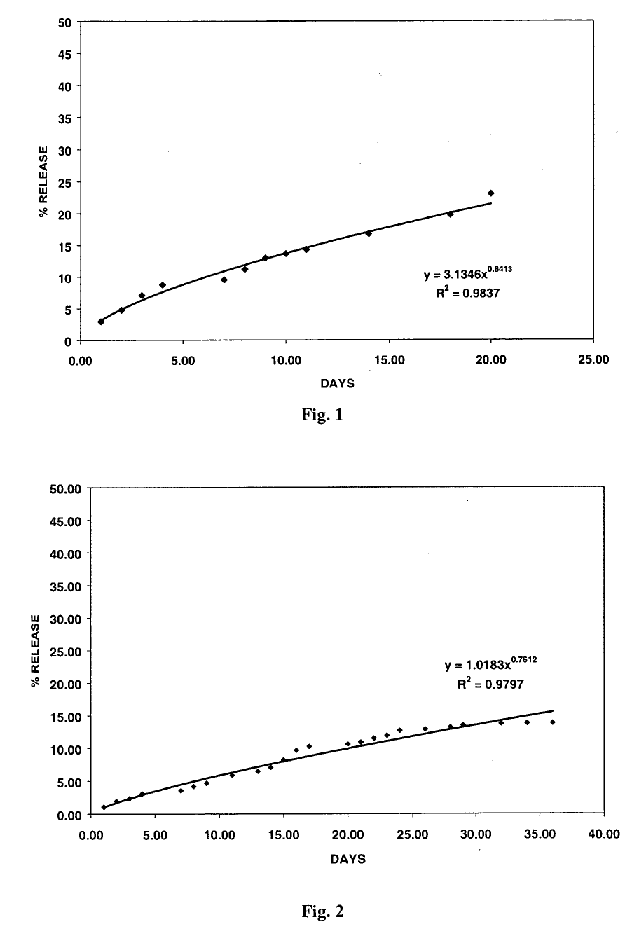

The dried piece of the graft was immersed in saline solution at 37° C. The solution was changed every 2-3 days. The solution was then analyzed by high performance liquid chromatography to determine the concentration of the agent eluted. A control piece of the agent loaded graft was exhaustively extracted with isopropanol and total loaded agent concentration was determined. From the total quantity of the loaded agent and the agent eluted from the graft at each time point, a release profile was constructed. FIG. 1, graphically depicts the release profile of Rapamycin loaded in a vascular access graft and eluted in vitro in saline.

### EXAMPLE 2

### Vascular Access Graft Loaded with Paclitaxel

Similarly Paclitaxel was also loaded onto Vectra® vascular access graft and release profile studied.

A 6 mm diameter graft was cut into two pieces. 23.6 mg (1% loading w/w graft) of Paclitaxel was dissolved in a minimum volume of ethanol (˜2 ml). The solution was placed in a glass trough and the graft halves deaired in the solution. All the solution was absorbed. Two control pieces were deaired in ethanol in the same manner. The grafts were oven dried at 80° C. for 60 minutes.

FIG. 2 graphically depicts the release profile for Paclitaxel loaded in a vascular access graft and eluted in vitro in saline.

### EXAMPLE 3

### Vascular Access Graft with Venous End Loaded with Rapamycin

A three layered graft was used. Although the two longitudinal ends of the graft are identical, after agent loading, the agent loaded end will be used as the venous end. A 2 cm length is identified at one end of the graft. A double lumen balloon catheter is inserted through the other end of the graft. The balloon is positioned so that the top edge of the balloon is in line with the 2 cm mark. A clamp is placed on the 2 cm mark that is towards the end of the graft. The graft is placed on a rocker so that the graft can be gently rocked from side to side.

The required amount of Rapamycin is weighed out in a vial (˜700 μg). A solution of the agent in 1 ml of ethyl acetate is prepared and transferred to a 2 ml syringe. The syringe is fixed to the lumen of the catheter and air pulled out of the space in the graft between the balloon end and the clamp. Let the syringe plunger to go. Due to the vacuum present in the space between the balloon and the clamp, the solution in the syringe is sucked into the lumen space in the graft. The graft is gently rocked so that the solution evenly coats the intimal surface of the graft. During the loading process, the solvent swells the polymer allowing the agent to diffuse into the polymer matrix.

The solvent evaporates through the middle layer. After about 30 minutes, the air is drawn out of the lumen pocket to place more agent solution into the pocket. This process is continued until all the solution is used up. The vial is rinsed with 0.5 ml of ethyl acetate and transferred to the syringe. The agent continues to be loaded into the inner layer as explained before. After completing loading of the agent in the inner layer (loading may also involve a bordering layer) of the graft, remove the balloon and the clamp.

Approximately 900 μg of the agent is weighed out in a vial. A solution of the agent in 1 ml of ethyl acetate is made. The adventitial layer of the graft is loaded at previously marked 2 cm length by simply placing the solution drop wise over the graft using a syringe or spraying the area with the solution. Each coat is applied after the previous coat is dried. After all the solution is applied to the graft, the graft is dried in a vacuum oven at room temperature for a minimum of 1 hour.

### EXAMPLE 4

### Stent Graft Loaded with Rapamycin

The stent grafts (6 mm dia, 7 crown, 7 ring) were loaded on a 7 mm balloon and the balloon was inflated to 10-12 atm. Rapamycin 1 mg was dissolved in 0.5 ml ethyl acetate. The solution was taken into a 0.5 ml syringe. 3-5 drops of the solution were added along the length of the stent graft. The balloon was rotated about 180° and 3-5 drops of the solution were added to the remaining part of the stent graft. The solvent is evaporated from the stent grafts for about 2-5 min, and the procedure is repeated until all the solution is added to the stent graft.

An additional 0.25 ml of fresh solvent is added to the Rapamycin vial and the solution is taken into the syringe. Continue adding the solution over the stent graft until all the solution is added. The stent graft is then air dried over the balloon for about 15 minutes and then removed from the balloon. The stent graft is dried in the vacuum oven for about an additional 45 minutes.

Distribution of Rapamycin in Stent Graft:

Each of the stent rings were separated by cutting the polymer between the rings. The stent rings containing the agent loaded polymer were extracted with 5 ml ethanol. The ethanol extract was analyzed by high performance liquid chromatography to quantify the amount of Rapamycin. The Rapamycin present in each of the stent rings was normalized to the weight of the polymer and plotted.

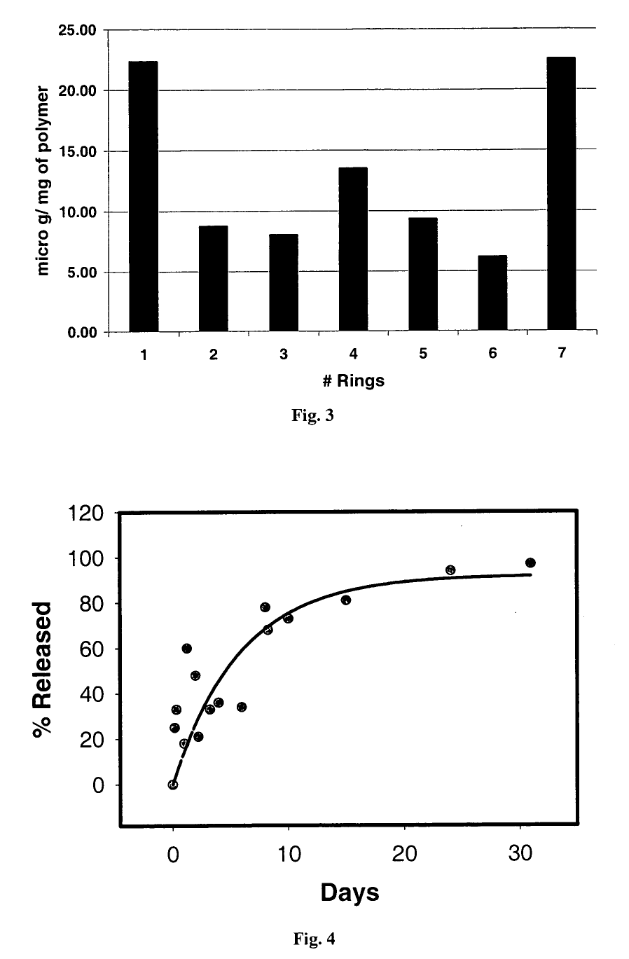

FIG. 3 graphically depicts the distribution of rapamycin at the rings of a stent graft.

Release Profile of Rapamycin in 4% Bovine serum Albumin Solution:

The stent grafts (6 mm diameter; 7 crown, 8 ring) were each loaded with 1 mg of Rapamycin. The stent grafts were cut into half and both halves were suspended in a vial containing 4% bovine serum albumin in saline solution (5 ml). The vials were placed in an incubator kept at 37° C. and the solution was gently agitated. The solution was changed every 3-4 days. Two halves of the stent grafts were removed from the solution at various time points and rinsed in water. The graft pieces were then extracted in ethanol and the ethanol extract was analyzed for remaining Rapamycin. From the quantity obtained at each time point and quantity loaded, a release profile was obtained. FIG. 4 graphically depicts experimental data demonstrating the release profile for rapamycin.

### EXAMPLE 5

### Polymer-paclitaxel Film

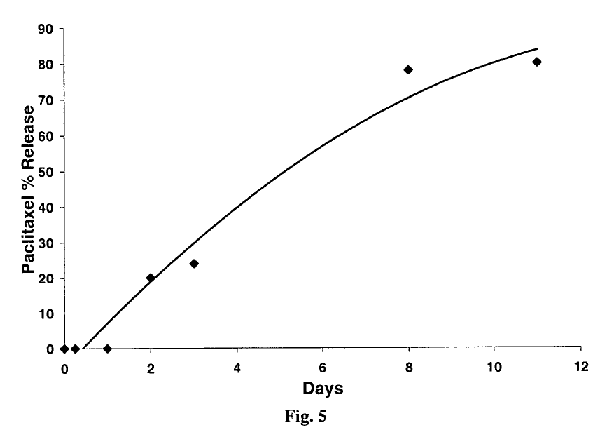

In this example, Paclitaxel was dissolved in DMAC (0.5 wt % to solids) and added to the polymer solution. The solution was then cast into a film. The film was cut into small pieces of known weight and suspended in 4% BSA solution. The solution was kept at 37° C. and slowly agitated. The solution was changed every 3-4 days. Samples were removed from the solution and rinsed in water. The samples were then extracted in ethanol and ethanol was analyzed for remaining Paclitaxel. FIG. 5 graphically depicts experimental data demonstrating the release profile of Paclitaxel from film.

1. A bioimplantable device comprising at least one region or layer for intimate contact with body tissue, said intimal layer or region either comprising or being in fluid communication with a portion of the device comprising a polyetherurethane;
the polyetherurethane being modified by admixture with a siloxane surface modifying additive;
at least some of the modified polyetherurethane portion containing a therapeutic agent.

2. The device of claim 1 wherein fewer than all polyetherurethane portions of the device contain therapeutic agent.

3. The device of claim 1 wherein all polyetherurethane portions contain therapeutic agent.

4. The device of claim 1 wherein the therapeutic agent is loaded on at least some but not all of the siloxane modified polyetherurethane portion of a region or layer.

5. The device of claim 1 wherein the therapeutic agent is loaded on all of the siloxane modified polyetherurethane portion of a region or layer.

6. The device of claim 1 adapted for service in an organ.

7. The device of claim 1 adapted for service in a tissue.

8. The device of claim 1 adapted for service as an anatomical support.

9. The device of claim 1 adapted for service as an arteriovenous shunt.

10. The device of claim 1 adapted for service as a stent.

11. The device of claim 1 adapted for service as a stent graft.

12. The device of claim 1 adapted for service as an endograft.

13. The device of claim 1 adapted for service as a vascular prosthesis.

14. The device of claim 1 adapted for service as a catheter.

15. The device of claim 1 wherein an agent soluble in siloxane modified polyetherurethane solution, is loaded in the range from about 0.001 to 40 weight percent of siloxane modified polyetherurethane.

16. The device of claim 1 wherein an agent soluble in siloxane modified polyetherurethane solution, is loaded in the range from about 0.001 to 30 weight percent of siloxane modified polyetherurethane.

17. The device of claim 1 wherein an agent soluble in siloxane modified polyetherurethane solution, is loaded in the range from about 0.001 to 20 weight percent of siloxane modified polyetherurethane.

18. The device of claim 1 wherein an agent soluble in siloxane modified polyetherurethane solution, is loaded in the range from about 0.001 to 10 weight percent of siloxane modified polyetherurethane.

19. The device of claim 1 wherein an agent soluble in siloxane modified polyetherurethane solution, is loaded in the range from about 0.001 to 5 weight percent of siloxane modified polyetherurethane.

20. The device of claim 1 wherein the loading is of an amount greater than about 10% of a systemically effective amount by weight of the composition.

21. The device of claim 1 wherein the loading is of an amount less than a systemically effective amount by weight of the composition.

22. The device of claim 1 wherein the loading is of an amount less than about 50% of a systemically effective amount by weight of the composition.

23. The device of claim 1 wherein the loading is of an amount less than about 40% of a systemically effective amount by weight of the composition.

24. The device of claim 1 wherein the loading is of an amount less than about 30% of a systemically effective amount by weight of the composition.

25. The device of claim 1 wherein the loading is of an amount less than about 20% of a systemically effective amount by weight of the composition.

26. The device of claim 1 wherein the loading is of an amount less than about 10% of a systemically effective amount by weight of the composition.

27. The device of claim 1 wherein the loading is of an amount greater than zero but less than about 5% of a systemically effective amount by weight of the composition.

28. The device of claim 1 wherein the loading is determined based on the loading of a layer.

29. The device of claim 1 wherein the loading is determined based on the loading of at least one, but fewer than all, layers.

30. The device of claim 1 wherein the loading is determined based on the loading of all layers.

31. The device of claim 1 wherein the polyetherurethane polymer of at least one layer comprises at least about 1 percent by weight of a polysiloxane-polyurethane copolymer surface modifying agent.

32. The device of claim 1 wherein the polyetherurethane polymer of at least one layer comprises at least from about 1 to about 5 percent by weight of a polysiloxane polyurethane copolymer surface modifying agent.

33. The device of claim 1 wherein the polyetherurethane polymer of at least one layer comprises from about 1 to about 40 percent by weight of a polysiloxane polyurethane copolymer surface modifying agent.

34. The device of claim 1 wherein said therapeutic agent is rapamycin.

35. The device of claim 1 wherein said therapeutic agent is paclitaxel.

36. The device of claim 1 wherein a plurality of therapeutic agents is loaded onto the device.

37. The device of claim 36 wherein the plurality of therapeutic agents are loaded onto different layers of the device.

38. The device of claim 36 wherein the plurality of therapeutic agents do not contact one another.

39. The device of claim 36 wherein the plurality of therapeutic agents are loaded onto the same layer of the device.

40. The device of claim 39 wherein at least two of the plurality of therapeutic agents do not physically contact one another.

41. A method of preventing or inhibiting development of hyperplasia comprising contacting a mammal with the prosthetic device of claim 1.

42. A method of localized delivery of a therapeutic agent to a target location within a mammal, comprising contacting a vessel within said mammal with the prosthetic device of claim 1.

43. A vascular graft comprising a generally tubular polyetherurethane and having two ends, said graft comprising:
an intimal layer comprising a substantially microporous polyetherurethane;
an intermediate layer comprising a substantially nonporous polyetherurethane; and
an adventitial layer comprising a substantially microporous polyetherurethane;
wherein the polyetherurethane of said layers may be the same or different; and
the polyetherurethane of at least one layer being modified by admixture with a siloxane surface modifying additive and
at least a portion of the polyetherurethane modified by admixture with siloxane containing polymer of at least one layer contains at least one therapeutic agent.

44. The graft of claim 43 wherein said therapeutic agent is loaded along the length of the graft.

45. The graft of claim 43 wherein the therapeutic agent is loaded at at least one end of the graft.

46. The graft of claim 43 adapted for service as an arteriovenous shunt.

47. The graft of claim 43 adapted for service as a stent.

48. The graft of claim 43 adapted for service as a stent graft.

49. The graft of claim 43 adapted for service as an endograft.

50. The graft of claim 43 adapted for service as a vascular prosthesis.

51. The graft of claim 43 adapted for service as an anatomical support.

52. The graft of claim 43 adapted for service as a catheter.

53. The graft of claim 43 wherein the therapeutic agent is loaded at the venous end of said graft while the arterial end is substantially free of therapeutic agent.

54. The graft of claim 43 wherein substantially all of said loading resides within about 10 cm from said venous end.

55. The graft of claim 43 wherein substantially all of said loading resides within about 5 cm from said venous end.

56. The graft of claim 43 wherein said therapeutic agent is loaded on the intimal and intermediate layer.

57. The graft of claim 56 wherein said therapeutic agent is loaded on the venous end of the graft while the arterial end is substantially free of said therapeutic agent.

58. The graft of claim 56 wherein said therapeutic agent is also loaded on said adventitial layer.

59. The graft of claim 43 wherein said therapeutic agent is loaded on the venous end of each of the three layers while the arterial end is substantially free of said therapeutic agent.

60. The graft of claim 43 wherein from about 1 nanogram to about 5000 mg of therapeutic agent is loaded onto the graft.

61. The device of claim 43 wherein an agent soluble in siloxane modified polyetherurethane solution, is loaded in the range from about 0.001 to 40 weight percent of siloxane modified polyetherurethane.

62. The device of claim 43 wherein an agent soluble in siloxane modified polyetherurethane solution, is loaded in the range from about 0.001 to 30 weight percent of siloxane modified polyetherurethane.

63. The device of claim 43 wherein an agent soluble in siloxane modified polyetherurethane solution, is loaded in the range from about 0.001 to 20 weight percent of siloxane modified polyetherurethane.

64. The device of claim 43 wherein an agent soluble in siloxane modified polyetherurethane solution, is loaded in the range from about 0.001 to 10 weight percent of siloxane modified polyetherurethane.

65. The device of claim 43 wherein an agent soluble in siloxane modified polyetherurethane solution, is loaded in the range from about 0.001 to 5 weight percent of siloxane modified polyetherurethane.

66. The device of claim 43 wherein the loading is of an amount greater than about 10% of a systemically effective amount by weight of the composition.

67. The graft of claim 43 wherein the loading is of an amount less than a systemically effective amount by weight of the composition.

68. The graft of claim 43 wherein the loading is of an amount less than about 50% of a systemically effective amount by weight of the composition.

69. The graft of claim 43 wherein the loading is of an amount less than about 40% of a systemically effective amount by weight of the composition.

70. The graft of claim 43 wherein the loading is of an amount less than about 30% of a systemically effective amount by weight of the composition.

71. The graft of claim 43 wherein the loading is of an amount less than about 20% of a systemically effective amount by weight of the composition.

72. The graft of claim 43 wherein the loading is of an amount less than about 10% of a systemically effective amount by weight of the composition.

73. The graft of claim 43 wherein the loading is of an amount greater than zero but less than about 5% of a systemically effective amount by weight of the composition.

74. The graft of claim 43 wherein the polyetherurethane polymer of at least one layer comprises at least about 1 percent by weight of a polysiloxane-polyurethane copolymer surface modifying agent.

75. The graft of claim 43 wherein the polyetherurethane polymer of at least one layer comprises at least from about 1 to about 5 percent by weight of a polysiloxane polyurethane copolymer surface modifying agent.

76. The graft of claim 43 wherein the polyetherurethane polymer of at least one layer comprises from about 1 to about 40 percent by weight of a polysiloxane polyurethane copolymer surface modifying agent.

77. The graft of claim 43 wherein said therapeutic agent is rapamycin.

78. The graft of claim 43 wherein said therapeutic agent is paclitaxel.

79. The graft of claim 43 wherein a plurality of therapeutic agents is loaded onto the graft.

80. The graft of claim 79 wherein the plurality of therapeutic agents are loaded onto different layers of the graft.

81. The graft of claim 79 wherein the plurality of therapeutic agents do not contact one another.

82. The graft of claim 79 wherein the plurality of therapeutic agents are loaded onto the same layer of the graft.

83. The graft of claim 82 wherein at least two of the plurality of therapeutic agents do not physically contact one another.

84. A method of preventing or inhibiting development of hyperplasia comprising contacting a mammal with the prosthetic graft of claim 43.

85. A method of localized delivery of a therapeutic agent to a target location within a mammal, comprising contacting a vessel within said mammal with the prosthetic graft of claim 43.

86. The method of claim 85 wherein said target location is substantially the proximal or distal anastomosis.

87. The method of claim 85 wherein said target location is substantially the arterial or the venous anastomosis.

88. A method of forming a prosthetic graft containing polyetherurethane and a therapeutic agent comprising contacting a prosthetic graft containing a polyetherurethane with a solution comprising a solvent and said therapeutic agent for a period of time sufficient to load said graft with a desired amount of therapeutic agent, wherein the solvent substantially swells the polymer allowing the agent to diffuse into the polymer matrix while said polyetherurethane is substantially insoluble in said solvent.

89. A method for forming a prosthetic graft which includes a therapeutic agent comprising:
mixing said therapeutic agent with a polyetherurethane polymer solution;
manufacturing the device;
applying the polymer to the intimal and adventitial surfaces of a polyethylene terephthalate or polytetrafluoroethylene graft.

90. A method for forming a coating comprising polyetherurethane polymer with siloxane based surface additives, said polymer loaded with a therapeutic agent.

91. The coating of claim 90 applied to a medical device.

92. The coating of claim 90 comprising rapamycin as a therapeutic agent.

93. The coating of claim 90 comprising paclitaxel as a therapeutic agent.

94. A biocompatible device comprising a blend of polyetherurethane polymer with siloxane based surface modifying additive, said blend being loaded with at least one therapeutic agent.

95. A device comprising a polyetherurethane having one or more layers, at least part of one layer comprising an admixture of siloxane surface modifying additive, and at least part of a layer comprising one or more therapeutic agents.

96. The device of claim 95 wherein the layers are anisotropically distributed throughout the device.
